(19) United States
(12) Patent Application Publication
(10) Pub. No.: US 2019/0046065 A1
(43) Pub. Date: Feb. 14, 2019

(54) Medical Electrode Providing Improved Uniformity of Current Density, Recovery Time and Durability

(71) Applicant: AED PAD LLC, Muskego, WI (US)

(72) Inventor: Robert A. Macur, East Troy, WI (US)

(21) Appl. No.: 16/059,930

(22) Filed: Aug. 9, 2018

Related U.S. Application Data
(60) Provisional application No. 62/544,859, filed on Aug. 13, 2017.
(60) Provisional application No. 62/544,862, filed on Aug. 13, 2017.

(51) Int. Cl. A61B 5/0408

(52) U.S. Cl. CPC A61B 5/04087; A61N 1/046

(57) ABSTRACT

An improved external medical electrode demonstrates uniform current distribution across its face, improved recovery time after delivering a defibrillation charge and improved ability to deliver pacing charges over extended periods. An ellipsoid, metal charge spreading layer is connected to a hydrogel layer by a carbon-containing polymer layer having on its hydrogel-contacting surface at least two layers of silver chloride-coated silver. A hyper-hydrosis inducing agent applied to the hydrogel layer on surface for contacting skin can substantially decrease skin impedance for improved operation.

### CROSS-REFERENCE TO RELATED APPLICATIONS

This application claims all benefits to and priority in U.S. Provisional Application Nos. 62/544,859 and 62/544,862, each filed on Aug. 13, 2017, the entirety of each of which is hereby expressly incorporated by reference herein.

### FIELD OF THE INVENTION

This invention relates generally to medical electrodes for use in treating or diagnosing conditions by the application or monitoring of electricity and, more specifically, to electrodes having improved uniformity of current density across the face of the electrode when delivering charge.

### BACKGROUND

Medical electrodes are commonly applied to a patient's skin to monitor electrical signals produced by the operation of internal organs, such as the heart, and to apply therapeutic electrical current through the skin to treat or control internal organs. For example, electrocardiograph (ECG or EKG) recording with medical electrodes is well known as is the use of medical electrodes to send external pacing signals to a heart through the skin. Medical electrodes also are used to apply relatively large electrical charges transcutaneously in urgent defibrillation situations.

Typically, electrical charges are applied or monitored through the skin by an electrode that contacts the skin with a hydrogel electrolyte layer. The hydrogel layer usually is held against the skin by an adhesive frame surrounding the hydrogel or by an external adhesive bandage.

It is important that medical electrodes that apply charges are evenly distributed across the surface of the electrode, especially in the case of defibrillation where the amount of electrical charge applied is large. Defibrillation electrodes are sized so as to distribute a relatively large charge evenly across a large skin area, avoiding concentrations of charge that would burn a patient's skin. Electrodes that do not provide even distribution of a defibrillation charge can result in a relatively large amount of charge entering the skin through a relatively small portion of the hydrogel surface of the electrode. Such events can result in serious burns to the patient.

The disclosure contained in U.S. published patent application 2014/0073896 A1 is illustrative of prior art efforts to provide more uniform energy distribution in an external medical electrode. That publication discloses a charge delivery wire sandwiched between a charge blocking layer and a charge distributing mat in a medical electrode structure.

In the disclosure contained in U.S. published patent application 2014/0288574 A1 a cause of burns to a patient's skin during defibrillation is identified as the relatively high impedance of the skin and is solved by placing defibrillation electrodes under the skin. The need for surgery to place electrodes subcutaneously is a disadvantage of the solution disclosed there.

In the disclosure contained in U.S. published patent application 2017/0106198 A1 a cause of burns to a patient's skin during defibrillation is identified as increased impedance resulting from the drying of hydrogel electrodes. In that disclosure the problem is solved by adding to a defibrillation system a circuit that monitors the hydration of the hydrogel electrodes and that triggers the addition of liquid to keep the electrodes hydrated. The need for complex circuitry is seen as a disadvantage of the solution disclosed in this publication.

In current medical devices a medical electrode can be used for both charge delivery and for monitoring signals produced by the body. For example, a medical electrode can be used to deliver a relatively massive charge for defibrillation and immediately thereafter used to monitor electrical signals from the heart being treated. Medical electrodes can take some time to recover from delivering a defibrillation charge before being useful to monitor heart signals through the skin. Typically the recovery time is about 20 seconds. A shorter recovery time would be an improvement.

In some situations a medical electrode that has been used in an emergency to defibrillate a heart and to then monitor the heart's performance is also called upon to deliver external pacing pulses transdermally to the heart while the heart patient awaits further medical attention. Pacing pulses can be delivered at the rate of 72 each minute for two hours. A rugged medical electrode that can withstand such repeated use over long periods of time would be welcome.

In addition, the impedance of skin is the largest source of electrical impedance in or out of the body. Typical electrical impedance of unprepared skin is about 30,000 to 50,000 Ohms. However, impedance of medical electrodes face-to-face is typically well under 40 Ohms. As a result, high currents applied through such electrodes through the skin have in some instances resulted in burns to the skin due to areas of high and low impedance through the skin.

In short, there is a need for a medical electrode that provides one or more of a more even distribution of charge, a quicker time to recover, sufficient ruggedness to remain useful over long periods of activity, and/or some reduction of the possibility of burn to the skin.

### SUMMARY OF THE INVENTION

These and other needs are satisfied by the improved medical electrode described herein, which includes an ellipsoid metal charge spreading layer that has first and second surfaces. The first surface of the charge spreading layer is connected by solder, substantially at its central point, to a source of electric current. The second surface of the charge spreading layer and a hydrogel layer are electrically connected by a carbon-containing conductive adhesive layer. The surface of the carbon containing conductive layer that contacts the hydrogel layer supports at least two coatings of silver-chloride coated silver. A hyper-hydrosis inducing agent applied to the hydrogel layer on surface for contacting skin can substantially decrease skin impedance for improved operation

The medical electrode of the present invention, which can be configured, for example, for ECG or defibrillation applications, provides improved uniformity of current density across the surface of the hydrogel layer, shorter recovery times and improved usefulness over extended periods of activity.

In one aspect, a hyper-hydrosis inducing agent or drug can be applied to the electrode where in contact with the skin, such as at the surface of the hydrogel layer. Hyper-hydrosis inducing agents have been shown to reduce skin impedance at the surface of a hydrogel in contact with the skin. Such compounds can elicit a hyper-hydrosis, or by common name, “sweating.” Exemplar drug compounds which may induce hyper-hydrosis include menthol and pilocarpine. Menthol has been available without a prescription for many years in numerous products, including for cold remedies. Pilocarpine has also been available, for instance, for use with newborn children to produce sweat for analyzing Sodium ions to screen for Cistic fibrosis. Pilocarpine has been shown to decrease skin impedance from 50,000 ohms to much less than 5,000 ohms at a pH of 3.5-4.5. The acrylic hydrogel used in most Defibrillator pads is buffered, for example, at a pH of 3.5.

These and other objects, advantages and aspects of the invention will become apparent from the following description. The particular objects and advantages described herein can apply to only some embodiments falling within the claims and thus do not define the scope of the invention. In the description, reference is made to the accompanying drawings which form a part hereof, and in which there is shown a preferred embodiment of the invention. Such embodiment does not necessarily represent the full scope of the invention and reference is made, therefore, to the claims herein for interpreting the scope of the invention.

### BRIEF DESCRIPTION OF THE DRAWINGS

Preferred exemplary embodiments of the invention are illustrated in the accompanying drawings in which like reference numerals represent like parts throughout, and in which:

FIG. 1 shows the medical electrode of the present invention in an expanded perspective view;

FIG. 2 shows an alternative aspect of the medical electrode of the present invention in an expanded perspective view;

FIG. 3 shows a medical electrode of the present invention implemented as an EKG electrode in a plan view; and

FIG. 4 shows a medical electrode of the present invention implemented as a defibrillation electrode in a plan view.

### DETAILED DESCRIPTION OF THE INVENTION

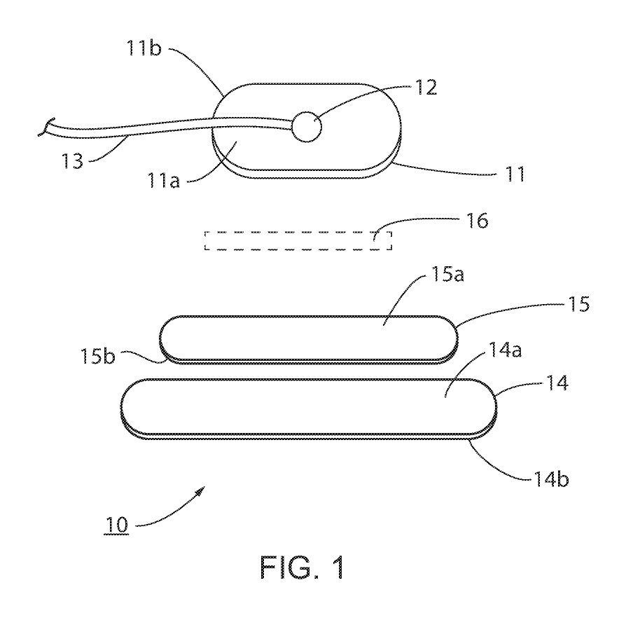

Referring now to FIG. 1, a medical electrode 10 includes an ellipsoid metal electrical charge-spreading layer 11. Charge spreading layer 11 may be formed from any electrically conductive metal including, for example, copper, aluminum, silver and alloys thereof. It is preferable that the metal is selected so that the charge spreading layer 11 is flexible in a substantially thin layer.

An oval spreading layer 11 made from copper was selected by comparative trials as the optimal material and shape for use in the present invention.

Although spreading layer 11 may be of any useful thickness, a thickness limited to about 0.005 inch (5 mils) has been found to be convenient for use in medical electrodes.

Spreading layer 11 has first surface 11a and second surface 11b opposing the first surface 11a. First surface 11a is connected at about its central point by a solder connection 12 to a source 13 of electric current. Sources of electric current in medical electrodes typically are insulated wires that deliver current and return signals to a control device such as, for example, a defibrillator or an ECG monitor. Source 13 in medical electrode 10 comprises a tinned copper wire.

It has been determined by comparative trials that both the material used for solder connection 12 and its location are important to the best performance of the present invention as a medical electrode. The material for use in solder connection 12 was selected to be lead-free because of its potential contact by medical patients. A variety of commonly available lead-free solder materials are useful; however, best results were obtained when a tin/silver/copper alloy was selected to form solder connection 12.

Second surface 11b of ellipsoid charge spreading layer 11 is electrically connected to a first surface 14a of hydrogel layer 14 by conductive layer 15. A second surface 14b of the hydrogel layer 14, for contacting the skin, opposes the first surface 14a of the hydrogel layer 14. Conductive layer 15 is, in the embodiment shown in FIG. 1, a carbon powder filled vinyl layer, such as PVP (Polyvinylpyrolidone), defining first surface 15a that is attached to second surface 11b of charge spreading layer 11, typically by a conductive adhesive material 16 Conductive layer 15 also defines a second surface 15b (opposing first surface 15a) that contacts the first surface 14a of hydrogel layer 14. Thickness of the conductive layer 15 could be limited to, for example, 4 micro inches. Thickness of the hydrogel layer 14 could be limited to, for example, 0.014 inch (14 mils).

Second surface 15b supports at least two coatings of silver chloride coated silver (Ag—AgCl). In a preferred embodiment, the first such coating comprises electroplated silver treated so as to give it a silver chloride coating. A second silver coating can be painted over the first such coating prior to the second coating being treated to have a silver chloride surface coating. Thickness of the two coatings of silver chloride coated silver could be limited to, for example, 0.005 inch (5 mils).

Flexible, polymeric materials filled with carbon powder to render them conductive are well known and commercially available. Hydrogel materials suitable for use in medical electrodes also are well known and commercially available.

Medical electrodes typically include an insulating backing or cover and a release liner covering the hydrogel surface, the release liner being removed just prior to applying the hydrogel surface to the skin of the patient being treated or diagnosed. Further, most medical electrodes include a means for adhering the hydrogel surface to the skin during use. The hydrogel layer in some commercially available medical electrodes is sometimes surrounded by an adhesive frame that is exposed when the release liner is removed, which adhesive frame can be used to adhere the medical electrode to the skin.

Such well-known materials and means for maintaining a medical electrode in contact with the skin can be used with the present invention and are not shown in FIG. 1.

The medical electrode of the present invention has been tested to determine its ability to provide a substantially uniform current across the face of the hydrogel surface by a proprietary method that is the subject of a commonly-assigned patent application filed on even date herewith and was found to provide an electrode surface having a uniform current density across its face when in use.

The medical electrode of the present invention also has been tested for recovery time following defibrillation use and was found to recover in 7 seconds compared with an average recovery time of 20 seconds for commercially available medical electrodes. The present medical electrode also has been tested for robustness by subjecting it to 10 v pulses having a duration of 20 ms at the rate of 170 pulses per minute for 60 minutes with outstanding post pulse results.

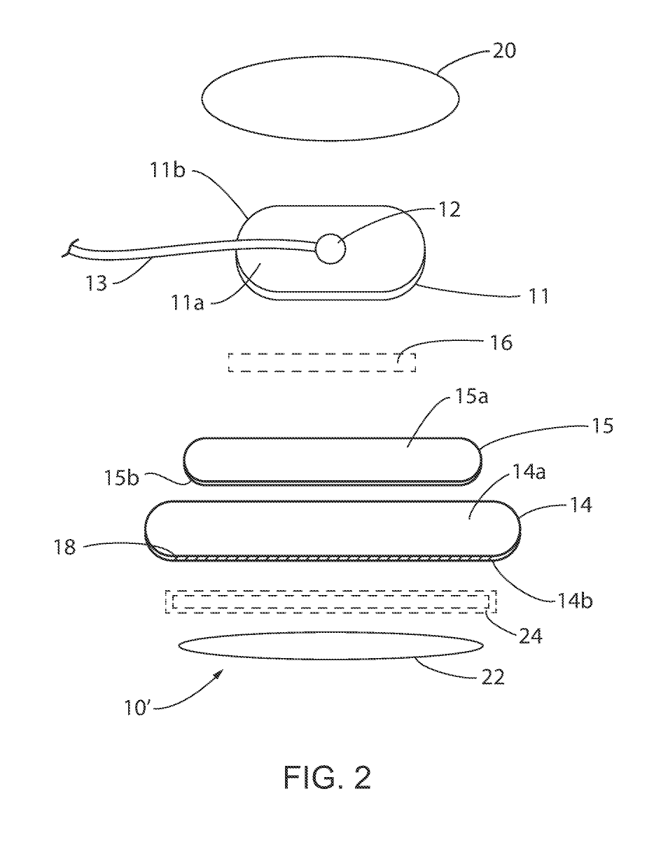

Referring now to FIG. 2, where like numerals refer to like parts throughout, a medical electrode 10′ according to another aspect of the invention includes a hyper-hydrosis inducing agent 18 applied to the second surface 14b of the hydrogel layer 14. The hyper-hydrosis inducing agent 18 could be pilocarpine or menthol, such as in an aqueous 5% solution. The hyper-hydrosis inducing agent 18 can elicit hyper-hydrosis, or by common name, “sweating,” to occur by the skin. As a result, the skin impedance under the electrode 10′ can substantially reduce, such as from 50,000 ohms to much less than 5,000 ohms, for improved conductivity while minimizing adverse effects with respect to the skin.

The electrode 10′ can include an insulating backing 20 or cover, which can substantially cover, electrically insulate and protect the first surface 11a of the spreading layer 11 and the solder connection 12, and a release liner 22, such as a mylar film, covering the second surface 14b of the hydrogel layer 14. The release liner can be easily removed just prior to applying the hydrogel surface to the skin of the patient being treated or diagnosed. Also, in one aspect, an adhesive frame 24 can surround the second surface 14b of the hydrogel layer 14 for improved adherence of the electrode 10′ to the skin (following removal of the release liner 22). Thickness of release liner 22 could be limited to, for example, 0.004 inch (4 mils).

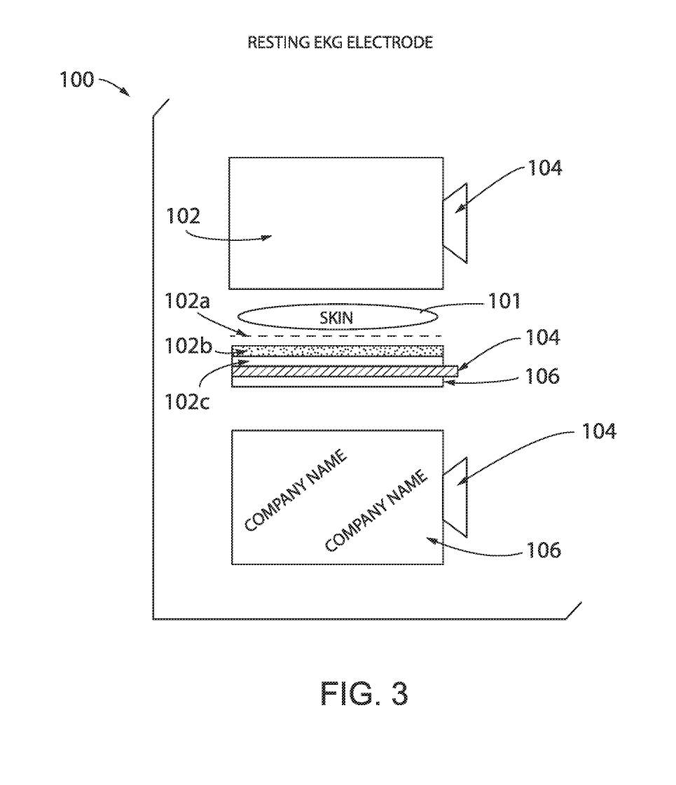

Referring now to FIG. 3, in one aspect, a medical electrode of the present invention can be implemented as a resting tab type EKG electrode 100 (without a lead wire, but rather, with an electrical clip being used to transmit a measurement signal) as shown and described (bottom, side and top). The EKG electrode 100 can include a composite layer 102 for contacting skin 101. The composite layer 102 can include: a menthol layer 102a, such as an aqueous 5% menthol (0.2 ml per square inch), closest to the skin 101; followed by a hydrogel layer 102b, such as polyvinylpyrolidone hydrogel coating (14 mils thick); followed by a conductive layer 102c, such as Ag—AgCl coating on conductive film (4 micro inches thick), furthest from the skin 101. The composite layer 102 can be placed over a carbon layer 104, such as a carbon conductive PVC film (4 mils thick). The carbon layer 104 can be covered by label tape 106 which could include a company name or logo. A release liner film, such as mylar film (4 mils thick), can be placed over the composite layer 102 until the EKG electrode 100 is ready to be used on the skin 101.

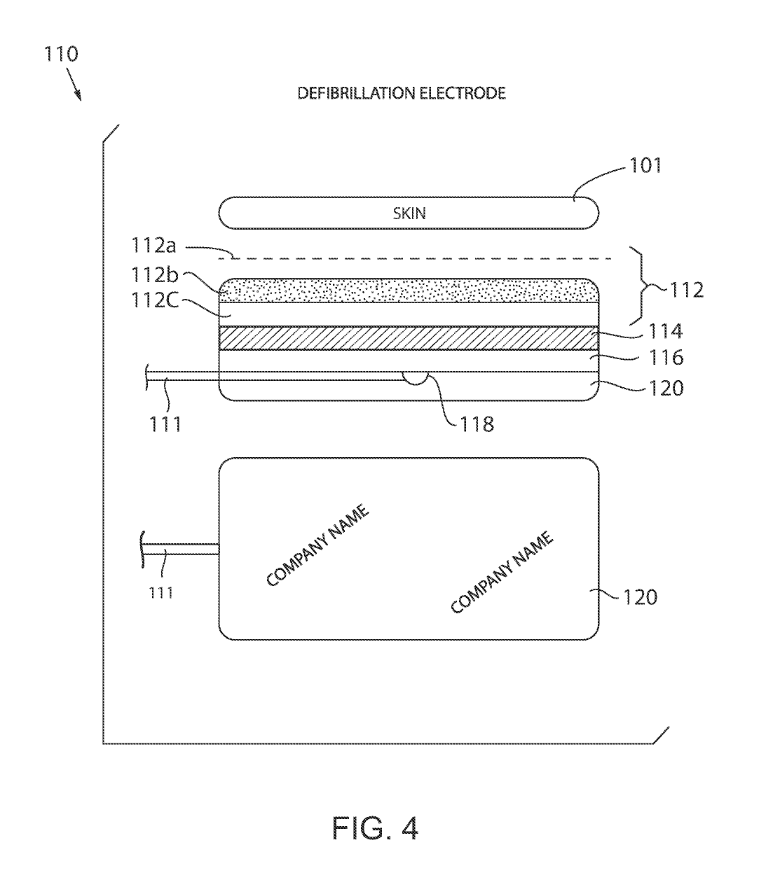

Referring to FIG. 4, in another aspect, a medical electrode of the preferred embodiments can be implemented specifically as a defibrillation electrode 110 (with a lead wire 111, capable of accommodating a defibrillation voltage) as shown and described (side and top). The defibrillation electrode 110 can include a composite layer 112 for contacting skin 101. The composite layer 112 can include: a menthol layer 112a, such as an aqueous 5% menthol (0.2 ml per square inch), closest to the skin 101; followed by a hydrogel layer 112b, such as polyvinylpyrolidone hydrogel coating (14 mils thick); followed by a conductive layer 112c, such as Ag—AgCl coating on conductive film (6 mils thick), furthest from the skin 101. The composite layer 112 can be placed over a carbon layer 114, such as a carbon conductive carbon film (4 mils thick). The carbon layer 114 can be covered by charge spreading layer 116 with a lead wire 111 affixed thereto via a solder connection 118. The charge spreading layer 116 can be covered by label tape 120 which could include a company name or logo. A release liner film, such as mylar film (4 mils thick), can be placed over the composite layer 112 until the defibrillation electrode 110 is ready to be used on the skin 101.

It is specifically intended that the present invention not be limited to the embodiments and illustrations contained herein and the claims should be understood to include modified forms of those embodiments including portions of the embodiments and combinations of elements of different embodiments as coming within the scope of the following claims. All of the publications described herein including patents and non-patent publications are hereby incorporated herein by reference in their entireties.

What is claimed is:

1. A medical electrode comprising:
(a) a substantially flat, ellipsoid, metallic current-spreading layer, the layer defining first and second surfaces;
(b) the first surface of the current-spreading layer being connected by solder at substantially its center point to a source of electric current;
(c) at least a major portion of the second surface of the current-spreading layer being attached by means of a conductive adhesive to the first surface of a carbon containing conductive layer, the second surface of the carbon containing conductive layer supporting at least two coatings of silver chloride coated silver, and
(d) a hydrogel layer contacting the second surface of the carbon containing conductive conductive layer so as to provide an operative electrode surface,
whereby improved uniformity of current density across the operative electrode surface, improved recovery time after delivering a defibrillation charge and improved ability to deliver pacing charges over extended periods are provided.

2. The medical electrode of claim 1 further comprising an insulating backing held against the first face of the metal layer by means of an insulating adhesive.

3. The medical electrode of claim 1 further comprising a releasable covering adhered to and protecting the hydrogel layer prior to use.

4. The medical electrode of claim 1 wherein the metal layer is copper.

5. The medical electrode of claim 1 wherein the source of electric current is copper wire.

6. The medical electrode of claim 1 wherein the source of electric current is soldered to the first surface of the current spreading layer substantially at the center of the spreading layer using a leadfree tin/copper/silver alloy solder.

7. The medical electrode of claim 1 wherein the carbon containing conductive layer comprises carbon powder filled PVP.

8. The medical electrode of claim 1 wherein silver in the first of the at least two coatings of silver chloride-coated silver is an electroplated silver.

9. The medical electrode of claim 1, wherein a first surface of the a hydrogel layer is contacting the second surface of the carbon containing conductive layer, and further comprising a hyper-hydrosis inducing agent applied to a second surface of the hydrogel layer.

10. The medical electrode of claim 9, wherein the hyper-hydrosis inducing agent is selected from the group of pilocarpine and menthol.

11. In a medical electrode having a hydrogel layer defining its operative surface, the improvement comprising an ellipsoid metal current spreading layer connected on its first face to a source of electric current, the source being affixed to the first face of the spreading layer with solder at substantially the central point thereof, the second face of the spreading layer being in electrical communication with the hydrogel layer through a carbon-containing conductive layer, substantially all of the second face of the spreading layer being held in contact with the conductive layer by a conductive adhesive and the face of the conductive layer in contact with the hydrogel layer supporting at least two coatings of silver chloride-coated silver, whereby improved uniformity of current density across the operative electrode surface, a shortened recovery time following delivery of a defibrillation charge and improved ability to deliver pacing charges over extended periods are provided.

12. The medical electrode of claim 11, wherein the current spreading layer is copper.

13. The medical electrode of claim 11, wherein the source of electric current is copper wire.

14. The medical electrode of claim 11, wherein the solder comprises a lead free tin/copper/silver alloy.

15. The medical electrode of claim 11, wherein the conductive layer comprises carbon powder filled vinyl polymer.

16. The medical electrode of claim 11, wherein silver in the first of the at least two coatings of silver chloride-coated silver is an electroplated silver.

17. A medical electrode comprising:
a substantially flat, ellipsoid, metallic current-spreading layer having opposing first and second surfaces;
solder at a central point of the first surface of the current-spreading layer for connecting to a source of electric current;
a carbon containing conductive layer having opposing first and second surfaces;
a conductive adhesive attaching the second surface of the current-spreading layer to the first surface of the carbon containing conductive layer;
at least two coatings of silver chloride coated silver applied to the second surface of the carbon containing conductive layer;
a hydrogel layer having opposing first and second surface, wherein the first surface of the hydrogel layer contact the second surface of the carbon containing conductive layer; and
a hyper-hydrosis inducing agent applied to the second surface of the hydrogel layer.

18. The medical electrode of claim 17, wherein the hyper-hydrosis inducing agent is selected from the group of pilocarpine and menthol.

19. The medical electrode of claim 17, further comprising an insulating backing held against the first surface of the current-spreading layer by an insulating adhesive.

20. The medical electrode of claim 17, wherein the carbon containing conductive layer comprises carbon powder filled PVP.
